(19) United States
(12) Patent Application Publication
(10) Pub. No.: US 2026/0240732 A1
(43) Pub. Date: Aug. 20, 2026

(54) Emulsion with a Dialkyl Carbonate, Coconut Oil Esters, Linear C15-C19 Alkanes, a Volatile Hydrocarbon-Based Oil and a Polyoxyethylenated Glycol Fatty Acid Ester Polymer

(71) Applicant: L'OREAL, Paris (FR)

(72) Inventors: Sonia EYRAUD, Chevilly Larue (FR); Estelle PRUD'HOMME, Chevilly Larue (FR); Bérengére PERRON, Chevilly Larue (FR); Marie NICOULEAU, Chevilly Larue (FR)

(73) Assignee: L'OREAL, Paris (FR)

(21) Appl. No.: 18/878,960

(22) PCT Filed: Jun. 19, 2023
(86) PCT No.: PCT/EP2023/066386

§ 371 Date: Dec. 26, 2024

(30) Foreign Application Priority Data
Jun. 28, 2022 (FR) FR2206454

(51) Int. Cl. A61K 8/06; A61K 8/31; A61K 8/37; A61Q 1/02

(52) U.S. Cl. CPC A61K 8/064; A61K 8/31; A61K 8/37; A61Q 1/02; A61K 2800/34

(57) ABSTRACT

The present application relates to a silicone-free, water/oil emulsion comprising at least A) a continuous oily phase comprising i) at least one C₁₂-C₂₂ dialkyl carbonate; and ii) at least one mixture of esters of C₈-C₁₀ carboxylic acid(s) and of fatty alcohols derived from coconut oil; and iii) a mixture of C₁₅-C₁₉ alkanes; and ii) at least one volatile hydrocarbon-based oil; and B) an aqueous phase dispersed in said oily phase; C) at least one emulsifying non-ionic surfactant of the polyoxyethylenated glycol fatty acid ester polymer type. The invention also relates to a process for coating keratin materials, more particularly for making up and/or caring for keratin materials, such as the skin, characterized in that it comprises the application, to the keratin materials, of a composition as defined above.

### TECHNICAL FIELD

The present invention is targeted at providing, for the field of caring for and/or making up keratin materials, in particular the skin, a new composition in the form of a water-in-oil emulsion (also called inverse emulsion) which is very particularly advantageous with regard to its technical performance qualities and the sensory feelings with which it provides the user when it is applied on these keratin materials and in particular on the skin.

Cosmetic compositions, for example foundations, are commonly used to give the skin an esthetic colour, but also to enhance the beauty of irregular skin, by making it possible to hide marks and dyschromias, to reduce the visibility of relief imperfections, such as pores and wrinkles, and to conceal spots and acne marks; In this regard, coverage is one of the main properties sought. In the cosmetics field, water-in-oil emulsions, also called inverse emulsions, are particularly appreciated by consumers in the field of foundations, sun protection products or moisturizing creams, with regard to their cosmetic properties, in particular with regard to their comfort on application, which is reflected in particular by an absence of or a strong reduction in the feeling of tightness, of drying and/or the tacky and/or greasy effect, in comparison with “direct” oil-in-water emulsions. These same inverse emulsions also make it possible to contain a large amount of fillers and/or pigments and to obtain good homogeneity during application, in comparison with direct emulsions. However, such compositions exhibit the disadvantage of being generally difficult to stabilize.

Consumers are also increasingly seeking cosmetic products with good environmental protection. It is known that the silicones present in numerous make up compositions on the cosmetics market are not entirely satisfactory from an environmental point of view.

Consumers particularly expect a fine and light texture during application, and also a soft and light deposit.

There remains the need to find new formulas for caring for and/or making up keratin fibres, in the form of a silicone-free water-In-oil emulsion having good coverage properties to correct imperfections with a uniform result, good sensory properties such as easy spreading, fineness in particular, lightness, softness when spreading, a non-greasy effect, a non-tacky and soft effect of the deposit after drying, good storage stability with respect to temperature and better environmental compatibility.

In the course of its research, the applicant has discovered, unexpectedly, that this objective can be achieved with a composition in the form of a silicone-free, water-in-oil emulsion comprising, notably in a physiologically acceptable medium:
A) a continuous oily phase comprising
i) at least one C₁₂-C₂₂ dialkyl carbonate; and
ii) at least one mixture of esters of C₈-C₁₀ carboxylic acid(s) and of fatty alcohols derived from coconut oil (INCI name: Coconut Alcohol); and
iii) a mixture of linear C₁₅-C₁₉ alkanes; and
ii) at least one volatile hydrocarbon-based oil; and
B) an aqueous phase dispersed in said oily phase;
C) at least one emulsifying non-ionic surfactant of the polyoxyethylenated glycol fatty acid ester polymer type.

This discovery forms the basis of the invention.

### SUBJECTS OF THE INVENTION

Thus, according to one of its aspects, the present invention relates to a silicone-free composition in the form of a water-in-oil emulsion, comprising, notably in a physiologically acceptable medium:
A) a continuous oily phase comprising
i) at least one C₁₂-C₂₂ dialkyl carbonate; and
ii) at least one mixture of esters of C₈-C₁₀ carboxylic acid(s) and of fatty alcohols derived from coconut oil (INCI name: Coconut Alcohol); and
iii) a mixture of linear C₁₅-C₁₉ alkanes; and
ii) at least one volatile hydrocarbon-based oil; and
B) an aqueous phase dispersed in said oily phase;
C) at least one emulsifying non-ionic surfactant of the polyoxyethylenated glycol fatty acid ester polymer type.

The invention also relates to a process for coating keratin materials, more particularly for making up and/or caring for keratin materials, such as the skin, characterized in that it comprises the application, to the keratin materials, of a composition as defined above.

### Definitions

In the context of the present invention, the term “keratin material” is understood in particular to mean the skin (body, face, area around the eyes), the lips, the eyelashes and the eyebrows. More particularly, the term “keratin material” is understood to mean the skin.

The term “physiologically acceptable” is intended to mean compatible with the skin and/or skin appendages, which has a pleasant colour, odour and feel and which does not cause any unacceptable discomfort (stinging or tautness) liable to discourage the consumer from using this composition.

For the purposes of the present invention, the term “water-in-oil emulsion”, also referred to as inverse emulsion, is understood to denote any composition constituted of a continuous oily phase in which the aqueous phase is dispersed in the form of droplets, so as to observe a mixture which is macroscopically homogeneous to the naked eye.

The term “silicone” is intended to mean any compound formed of a silicon-oxygen chain —Si—O—Si—O—Si—O in which organic groups attach to the silicone atoms.

The term “silicone-free composition” is understood to mean any composition containing less than 1.0% by weight of silicone compound, indeed even less than 0.5% by weight, indeed even less than 0.1% by weight, relative to the total weight of the composition, indeed even devoid of silicone compound.

### Continuous Oily Phase

A composition in accordance with the present invention comprises a continuous oily phase containing
i) at least one C₁₂-C₂₂ dialkyl carbonate; and
ii) at least one mixture of esters of a C₈-C₁₀ carboxylic acid and of fatty alcohols derived from coconut oil (INCI name: Coconut Alcohol); and
iii) a mixture of linear C₁₅-C₁₉ alkanes; and
iv) at least one volatile hydrocarbon-based oil.

The term “oil” means any fatty substance that is in liquid form at ambient temperature (20-25° C.) and atmospheric pressure (760 mmHg).

A composition of the invention can comprise an oily phase in a content ranging from 15% to 5%, in particular from 25% to 40% by weight, relative to the total weight of the composition.

C₁₂-C₂₂ Dialkyl Carbonate

The oily phase of the composition according to the invention comprises i) at least one C₁₂-C₂₂ dialkyl carbonate of formula R₁COOR₂ in which R₁ and R₂, independently, denote a linear or branched alkyl radical containing from 1 to 16 atoms such that the total number of carbon atoms present in the R₁ and R₂ radicals is from 12 to 22.

Among the C₁₂-C₂₂ dialkyl carbonates, mention may be made of
di-2-ethylhexyl carbonate (C₁₇) such as the products sold under the trade name Tegosoft DEC® by Evonik Nutrition & Care GmbH;
dicaprytyl carbonate (C₁₇) such as the products sold under the trade names Cetiol CC® (BASF Corporation) and OriStar DCC® (Orient Stars LLC);
a mixture of C₁₄-C₁₅ dialkyl carbonates (INCI name: C14-C15 Dialkyl Carbonate) such as the products sold under the trade names Lialcarb SR-1000/R® by Enichem SpA and OrdStar C14-15 DAC® by Orient Stars LLC;
dipropylheptyl carbonate (C₂₁) such as the products sold under the trade name Cetiol 4 All® by BASF.

According to one particularly preferred form, the dialkyl carbonate is dicaprylyl carbonate.

The dialkyl carbonate(s) i) is (are) preferably present in the composition of the invention in contents ranging from 5% to 20% by weight, preferably from 7% to 18% by weight relative to the total weight of said composition.

Ester of a C₈-C₁₀ Carboxylic Acid and of a Mixture of Alcohols Derived from Coconut Oil

The oily phase of the composition according to the invention comprises ii) at least one mixture of esters of C₈-C₁₀ carboxylic acid(s) and of fatty alcohols derived from coconut oil (INCI name: Coconut Alcohol).

By way of example, mention may be made of the mixture of esters of caprylic acid (C₈) and of coconut oil fatty alcohols, having the INCI name Coco Caprylate with the formula below

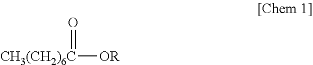

In which R denotes a coconut oil fatty alcohol residue. It may be sold under the trade name Cetiol C5® by BASF.

According to one particular embodiment of the invention, use will be made of a mixture of esters of caprylic acid (C₈) and of capric acid (C₁₀) and of coconut oil fatty alcohols, having the INCI name Coco Caprylate/Caprate. It may be sold under the following trade names
AEC Coco-Caprylate/Caprate® (A&E Connock Perfumery & Cosmetics Ltd);
Captex 170® (Abitec Corporation);
Cetiol C 5C®, Cetiol LCO (BASF Corporation);
Cremer COOR Coco 810® (IOI Oleo GmbH);
Dub 810 C® (Stearineries Dubois et fils);
Lanol 2681® (SEPPIC):
Pelemol CCC® (“Phoenix Chemical Inc);
Unimul-LC® and Unitolate LC® (Universal Presery-A-Chem Inc.).

The mixture ii) of esters of C₈-C₁₀ carboxylic acid(s) and of fatty alcohols derived from coconut oil is preferably present in the composition of the invention in contents ranging from 2% to 20% by weight, preferably from 4% to 15% by weight relative to the total weight of the composition.

Mixtures of Linear C₁₅-C₁₉ Alkanes

The oily phase of the composition according to the invention comprises iii) at least one mixture of linear C₁₅-C₁₉ alkanes such as the products sold under the references Emogreen L19® and Emosmart L19® from SEPPIC.

According to one preferred form of the invention, the amount of the mixture iii) of linear C₁₅-C₁₉ alkanes is less than or equal to 7% by weight relative to the total weight of the composition.

The mixture iii) of linear C₁₅-C₁₉ alkanes is preferably present in the composition of the invention in contents ranging from 1% to 12% by weight, preferably from 1% to 10% by weight relative to the total weight of the composition.

### Volatile Hydrocarbon-Based Oil

The oil phase of the composition according to the invention comprises iv) at least one volatile hydrocarbon-based oil.

The term “hydrocarbon-based oil” is understood to mean an oil containing mainly carbon and hydrogen atoms and possibly one or more functions chosen from hydroxyl, ester, ether or carboxyl functions.

For the purposes of the invention, the term “volatile oil” refers to any oil that is capable of evaporating on contact with the skin in less than one hour, at ambient temperature and atmospheric pressure. The volatile oil is a volatile cosmetic compound, which is liquid at ambient temperature, notably having a non-zero vapour pressure, at ambient temperature and atmospheric pressure, notably having a vapour pressure ranging from 2.66 Pa to 40 000 Pa, in particular ranging from 2.66 Pa to 13 000 Pa and more particularly ranging from 2.66 Pa to 1300 Pa.

The volatile hydrocarbon-based oils iv) that may be used in the compositions according to the invention may be chosen from branched C₈-C₁₆ alkanes.

Mention may notably be made, as C₈-C₁₆ isoalkanes of petroleum origin (also known as isoparaffins), for instance isododecane (also known as 2,2,4,4,6-pentamethylheptane), isodecane, isohexadecane and, for example, the oils sold under the trade name Isopar® or Permethyl®.

Mention may also be made of branched C₈-C₁₆ esters such as isohexyl neopentanoate. Other volatile hydrocarbon-based oils, for instance petroleum distillates, notably those sold under the name Shell Solt® by Shell, may also be used.

The volatile hydrocarbon-based oils that may be used in the compositions according to the invention may be chosen from volatile linear alkanes comprising from 6 to 14 carbon atoms.

Mention may be made, by way of example of linear alkanes suitable for the invention, of the alkanes described in the patent applications WO 2007/068371 and WO 2008/155059 of Cognis (mixtures of different alkanes differing by at least one carbon). These alkanes are obtained from fatty alcohols, themselves obtained from copra oil or palm oil.

Mention may be made, by way of example of linear C₆-C₁₄ alkanes suitable for the invention, of n-hexane (C₆), n-heptane (C₇), n-octane (C₈), n-nonane (C₉), n-decane (C₁₀), n-undecane (C₁₁), n-dodecane (C₁₂), n-tridecane (C₁₃), n-tetradecane (C₁₄) and mixtures thereof.

Mention may notably be made of n-dodecane (C₁₂) and n-tetradecane (C₁₄) sold by Sasol under the respective references Parafol 12-97® and Parafol 14-97®, and also mixtures thereof.

According to another embodiment, a mixture of n-dodecane and n-tetradecane is used. It is in particular possible to use the dodecane/tetradecane mixture in an 85/15 weight ratio sold by Biosynthis under the reference Vegelight 1214®.

According to yet another embodiment, use is made of a mixture of volatile linear C₉-C₁₂ alkanes of INCI name: C9-12 Alkane such as the product sold by Biosynthis under the reference Vegelight Silk®.

According to yet another embodiment, use is made of a mixture of n-undecane (C₁₁) and of n-tridecane (C₁₃) as obtained in Examples 1 and 2 of patent application WO 2008/155059 from Cognis and the product sold under the trade name Cetiol Ultimate® by BASF.

According to one particularly preferred embodiment, the volatile hydrocarbon-based oil is chosen from branched C₈-C₁₆ alkanes, and more particularly is isododecane.

The volatile hydrocarbon-based oil(s) i) are preferably present in the composition of the invention in contents ranging from 2% to 20% by weight and preferably from 3% to 15% by weight relative to the total weight of said composition.

Non-Ionic Surfactant which is a Polyoxyethylenated Glycol Fatty Acid Ester Polymer

The composition according to the invention comprises at least one emulsifying non-Ionic surfactant of the polyoxyethylenated glycol fatty acid ester polymer type.

For the purposes of the present invention, the term “emulsifying surfactant” is understood to mean an amphiphilic surfactant compound, that is to say one which exhibits two parts of different polarity. Generally, one is lipophilic (soluble or dispersible in an oily phase). The other is hydrophilic (soluble or dispersible in water). Emulsifying surfactants are characterized by the value of their HLB (Hydrophilic Lipophilic Balance), the HLB being the ratio of the hydrophilic part to the lipophilic part in the molecule. The term “HLB” is well known to those skilled in the art and is described, for example, in “The HLB System. A Time-Saving Guide to Emulsifier Selection” (published by ICI Americas Inc.; 1984). For emulsifying surfactants, the HLB generally ranges from 3 to 8 for the preparation of W/O emulsions. The HLB of the surfactant(s) used according to the invention can be determined by the Griffin method or the Davies method.

The emulsifying non-Ionic surfactant of the polyoxyethylenated glycol fatty acid ester polymer type has an HLB<8.

The fatty acid ester of said polymer is polyhydroxylated. In particular, this polymer is a block polymer, preferably of ABA structure, comprising poly(hydroxylated ester) blocks and polyethylene glycol (or polyoxyethylene) blocks.

The fatty acid ester of said emulsifying polymer as defined above generally has a chain comprising from 12 to 20 carbon atoms and preferably from 14 to 18 carbon atoms. The esters may be chosen in particular from oleates, palmitates or stearates.

The polyethylene glycol blocks of said emulsifying polymer as defined above preferably comprise from 4 to 50 mol of ethylene oxide and more preferably from 20 to 40 mol of ethylene oxide.

A compound that is particularly suitable for preparing the compositions of the invention is polyethylene glycol dipolyhydroxystearate containing 30 mol of ethylene oxide, having the INCI name: PEG-30 Polyhydroxystearate, such as the product sold under the trade reference Cithrol DPHS-SO-(MV) by Croda.

The polymer of polyoxyethylenated fatty acid ester of glycol is preferably present in the composition in contents ranging from 0.1% to 10% by weight, more preferentially from 1% to 8% by weight relative to the total weight of the composition.

### Aqueous Phase

The aqueous phase comprises water and optionally water-soluble or water-miscible ingredients, such as water-soluble solvents.

A water suitable for the invention can be a floral water, such as cornflower water, and/or a mineral water, such as Vittel water, Lucas water or La Roche-Posay water, and/or a thermal water.

Among the water-soluble solvents that may be present in the aqueous phase, mention may be made of C₂-C₄ monoalcohols such as ethanol, propanol, isopropanol or butanol. Mention may also be made of polyols such as glycerol, propanedlol, pentylene glycol, butylene glycol, caprylyl glycol, ethylhexyglyceryl, propylene glycol and mixtures thereof.

A composition of the invention can comprise water in a content varying from 20% to 50% by weight and even more preferably from 30% to 45% by weight, relative to the total weight of the composition.

### Pulverulent Colorants

According to one particular form of the invention, the composition according to the invention also comprises at least one pulverulent colorant.

The pulverulent colorants can be chosen from mineral pigments, organic pigments, pearlescent agents and mixtures thereof.

The term “pigments” means white or coloured, mineral or organic particles, which are Insoluble in an aqueous medium, and which are intended to colour and/or opacify the resulting composition and/or deposit. These pigments may be white or coloured, and mineral and/or organic.

According to a specific embodiment, the pigments used according to the invention are chosen from mineral pigments.

The term “mineral pigment” is understood to mean any pigment which satisfies the definition in Ullmann's Encyclopedia in the chapter “Pigments, Inorganic”. Mention may be made, among the mineral pigments of use in the present invention, of zirconium or cerium oxides, and also zinc, iron (black, yellow or red) or chromium oxides, manganese violet, ultramarine blue, chromium hydrate and ferric blue, titanium dioxide, or metal powders, such as aluminum powder and copper powder. The following mineral pigments may also be used: Ta₂O₅, Ti₃O₅, Ti₂O₃, TiO, ZrO₂ as a mixture with TiO₂, ZrO₂, Nb₂O₅, CeO₂ or ZnS.

The size of the pigment of use in the context of the present invention is generally greater than 100 nm and can range up to 10 μm, preferably from 200 nm to 5 μm and more preferentially from 300 nm to 1 μm.

According to a specific form of the invention, the pigments exhibit a size characterized by a D[50] of greater than 100 nm and which can range up to 10 μm, preferably from 200 nm to 5 μm and more preferentially from 300 nm to 1 μm.

The sizes are measured by static light scattering using a commercial MasterSizer 3000® particle size analyzer from Malvern, which makes it possible to determine the particle size distribution of all of the particles over a wide range which may extend from 0.01 μm to 1000 μm. The data are processed on the basis of the standard Mie scattering theory. This theory is the most suitable for size distributions ranging from the submicronic to multimicronic; it makes it possible to determine an “effective” particle diameter. This theory is described in particular in the publication by Van de Hulst, H. C., Light Scattering by Small Particles, Chapters 9 and 10, Wiley, New York, 1957.

D[50] represents the maximum size exhibited by 50% by volume of the particles.

According to a specific form of the invention, the mineral pigment comprises a lipophilic or hydrophobic coating; the latter is preferably present in the oily phase of the composition according to the invention.

According to a specific embodiment of the invention, the pigments can be coated according to the invention with at least one compound chosen from metal soaps; N-acylamino acids or their salts; lecithin and its derivatives; isopropyl triisostearyl titanate; isostearyl sebacate; natural plant or animal waxes; polar synthetic waxes; fatty esters; phospholipids; and mixtures thereof.

According to a preferential embodiment, the pigments can be coated according to the invention with an N-acylamino acid or one of its salts which can comprise an acyl group having from 8 to 22 carbon atoms, such as, for example, a 2-ethylhexanoyl, caproyl, lauroyl, myristoyl, palmitoyl, stearoyl or cocoyl group.

The amino acid can, for example, be lysine, glutamic acid or alanine. The salts of these compounds can be the aluminium, magnesium, calcium, zirconium, zinc, sodium or potassium salts. Thus, according to one particularly preferred embodiment, the pigments can be coated with an N-acylamino acid derivative which can in particular be a glutamic acid derivative and/or one of its salts, and more particularly a stearoyl glutamate, such as, for example, aluminium stearoyl glutamate. Mention may be made, as examples of pigments treated with aluminum stearoyl glutamate, of titanium dioxide pigments and black, red and yellow iron oxide pigments sold under the trade name Nai® by Miyoshi Kasei.

According to a preferential embodiment, the pigments can be coated according to the invention with isopropyl triisostearyl titanate. Mention may be made, as examples of pigments treated with isopropyl titanium trilsostearate (ITT), of those sold under the trade names BWBO-I2® (Iron Oxide CI77499 and Isopropyl Titanium Triisostearate), BWYO-I2® (Iron Oxide CI77492 and Isopropyl Titanium Triisostearate) and BWRO-I2® (Iron Oxide CI77491 and Isopropyl Titanium Triisostearate) by Kobo.

The pigments which can be used according to the invention can also be organic pigments.

The term “organic pigment” refers to any pigment that satisfies the definition in Ullmann's encyclopedia in the chapter on organic pigments. The organic pigment can in particular be chosen from nitroso, nitro, azo, xanthene, quinoline, anthraquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

The organic pigment(s) may be chosen, for example, from carmine, carbon black, aniline black, melanin, azo yellow, quinacridone, phthalocyanine blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives as described in patent FR 2 679 771.

These pigments can also be in the form of composite pigments as are described in the patent EP1184426. These composite pigments can be composed in particular of particles comprising an inorganic core at least partially covered with an organic pigment and at least one binder providing the fixing of the organic pigments to the core.

The pigment can also be a lake. The term “lake” is understood to mean insolubilized dyes adsorbed on insoluble particles, the assembly thus obtained remaining insoluble during use.

The inorganic substrates onto which the dyes are adsorbed are, for example, alumina, silica, calcium sodium borosilicate or calcium aluminum borosilicate and aluminum.

Among the organic dyes, mention may be made of cochineal carmine. Mention may also be made of the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green 5 (CI 61 570), D&C Yellow 10 (CI 77 002), D&C Green 3 (CI 42 053), D&C Blue 1 (CI 42 090).

Mention may be made, by way of an example of a lake, of the product known under the name D&C Red 7 (CI 15 850:1).

Preferably, the composition according to the invention comprises at least one pulverulent colorant of mineral pigment type, in particular chosen from metal oxides, and more particularly chosen from titanium dioxides and iron oxides, which are or coated or uncoated, and mixtures thereof.

The pearlescent agents can be chosen from white pearlescent pigments, such as mica covered with titanium or with bismuth oxychloride, coloured pearlescent pigments, such as titanium oxide-coated mica with iron oxides, titanium oxide-coated mica in particular with ferric blue or chromium oxide, or titanium oxide-coated mica with an organic pigment of the abovementioned type, and also pearlescent pigments based on bismuth oxychloride.

Preferably, the pulverulent colorant(s) is (are) preferably present in the composition in a content ranging from 0.5% to 30% by weight, preferably from 1% to 25% by weight, more particularly from 3% to 20% by weight, relative to the total weight of the composition.

### Cosmetic Compositions

The present invention also relates to a cosmetic composition comprising, in a physiologically acceptable medium, a composition as defined above.

The physiologically acceptable medium is generally adapted to the nature of the support onto which the composition has to be applied, and also to the appearance under which the composition has to be packaged.

The compositions according to the invention can additionally comprise additives commonly used in care and/or makeup products, such as:
active agents such as vitamins, for example vitamins A, E, C and B3, adenosine, hyaluronic acid and its salts;
ceramides;
inorganic UV-screening agents
additional colorants;
fillers;
hydrophilic gelling agents;
lipophilic gelling agents;
fragrances;
preserving agents;
and mixtures thereof.

It is a matter of routine operations for those skilled in the art to adjust the nature and the amount of the additives present in the compositions in accordance with the invention so that the cosmetic properties desired for these are not thereby affected.

### Additional Colorants

A composition according to the invention can additionally comprise at least one additional water-soluble or fat-soluble colorant and preferably in a proportion of at least 0.01% by weight, relative to the total weight of the composition.

For obvious reasons, this amount is liable to vary significantly with regard to the desired intensity of the colour effect and to the colour intensity provided by the colorants under consideration, and its adjustment clearly falls within the competence of those skilled in the art.

The additional colorants suitable for the invention can be fat-soluble.

For the purposes of the invention, the term “liposoluble colorant” means any natural or synthetic, generally organic compound, which is soluble in an oily phase or in solvents that are miscible with a fatty substance, and which is capable of imparting colour.

As liposoluble dyes that are suitable for use in the invention, mention may notably be made of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

The additional colorants suitable for the invention can be water-soluble.

For the purposes of the invention, a “water-soluble colorant” is understood to mean any natural or synthetic, generally organic compound, which is soluble in an aqueous phase or water-miscible solvents and which is capable of imparting colour.

As water-soluble dyes that are suitable for use in the invention, mention may be made notably of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophyllin, methylene blue, anthocyanins (enocianin, black carrot, hibiscus, elder), caramel and riboflavin.

### Inorganic UV Screening Agents

According to one particular form of the invention, the composition also comprises at least one inorganic UV-screening agent

The inorganic UV-screening agents used in accordance with the present invention are metal oxide pigments. More preferentially, the inorganic UV-screening agents of the invention are metal oxide particles having a mean elementary particle size of less than or equal to 0.5 μm, more preferentially of between 0.005 and 0.5 μm, more preferentially still of between 0.01 and 0.2 μm, better still between 0.01 and 0.1 μm and more particularly between 0.015 and 0.05 μm.

They may be chosen in particular from titanium oxide, zinc oxide, iron oxide, zirconium oxide and cerium oxide, or mixtures thereof.

Such coated or uncoated metal oxide pigments are described in particular in patent application EP-A-0 518 773. Commercial pigments that may be mentioned include the products sold by the companies Sachtieben Pigments, Tayca, Merck and Degussa.

The metal oxide pigments may be coated or uncoated.

The coated pigments are pigments which have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or aluminium), polyethylene, proteins (collagen, elastin), alkanolamines, silicon oxides, metal oxides or sodium hexametaphosphate.

The coated pigments are more particularly titanium oxides coated:
with silica, such as the product Sunveil® from Ikeda,
with silica and iron oxide, such as the product Sunveil F® from Ikeda,
with silica and alumina, such as the products Microtitanium Dioxide MT 500 SA® and Microtitanium Dioxide MT 100 SA from Tayca and Tioveil from Tioxide,
with alumina, such as the products Tipaque TTO-55 (B)® and Tipaque TTO-55 (A)® from Ishihara and UVT 14/4 from Sachtleben Pigments,
with alumina and aluminium stearate, such as the products Microtitanium Dioxide MT 100 T®, Microtitanium Dioxide MT-100 T V®, MT 100 TX®, MT 100 Z® and MT-01® from Tayca, the products Solaveil CT-10 W® and Solaveil CT 100® from Uniqema and the product Eusolex T-AVO® from Merck,
with silica, alumina and alginic acid, such as the product MT-100 AQ® from Tayca,
with alumina and aluminium laurate, such as the product Microtitanium Dioxide MT 100 S® from Tayca,
with iron oxide and iron stearate, such as the product Microtitanium Dioxide MT 100 F® from Tayca,
with zinc oxide and zinc stearate, such as the product BR 351® from Tayca,
with triethanolamine, such as the product STT-65-S from Titan Kogyo,
with stearic acid, such as the product Tipaque TTO-55 (C)® from Ishihara,
with sodium hexametaphosphate, such as the product Microtitanium Dioxide MT 150 W® from Tayca,
TiO₂ treated with octyltrimethylsilane, sold under the trade name T 805® by Degussa Silices,
TiO₂ coated with triethylhexanoin, aluminium stearate and alumina, sold under the trade name Solaveil CT-200-LQ-(WD) by Croda,
TiO₂ coated with aluminium stearate, with alumina and with silicone sold under the trade name Solaveil CT-12W-LQ-(WD) by Croda,
TiO₂ coated with lauroyl lysine sold by Daito Kasei Kogyo under the name LL 5 Titanium Dioxide CR 50,
TiO₂ coated with C9-15 fluoroalcohol phosphate and with aluminium hydroxide sold by Daito Kasei Kogyo under the name PFX-5 TiO2 CR-50.

Mention may also be made of TiO₂ pigments doped with at least one transition metal, such as iron, zinc or manganese and more particularly manganese. Preferably, said doped pigments are in the form of an oily dispersion. The oil present in the oily dispersion is preferably chosen from triglycerides, including those of capric/caprylic acids. The oily dispersion of titanium oxide particles can additionally comprise one or more dispersing agents, such as, for example, a sorbitan ester, such as sorbitan isostearate, a polyoxyalkylenated glycerol fatty acid ester, such as Tr-PPG-3 Myristyl Ether Citrate and Polyglyceryl-3 Polyricinoleate. Preferably, the oily dispersion of titanium oxide particles comprises at least one dispersing agent chosen from polyoxyalkylenated glycerol fatty acid esters. Mention may be made more particularly of the oily dispersion of TiO2 particles doped with manganese in capric/caprylic acid triglyceride in the presence of Tri-PPG-3 myristyl ether citrate and polyglyceryl-3 polyricinoleate and sorbitan isostearate having the INCI name: titanium dioxide (and) TRI-PPG-3 myristyl ether citrate (and) polyglyceryl-3 ricinoleate (and) sorbitan isostearate, for instance the product sold under the trade name Optisol TD50® by Croda.

The uncoated titanium oxide pigments are sold, for example, by Tayca under the trade names Microtitanium Dioxide MT 500 B or Microtitanium Dioxide MT 600 B®, by Degussa under the name P 25, by Wackher under the name Transparent titanium oxide PW®, by Miyoshi Kasel under the name UFTR®, by Tomen under the name ITS® and by Tioxide under the name Tioveil AQ.

The uncoated zinc oxide pigments are, for example:
those sold under the name Z-Cote by Sunsmart;
those sold under the name Nanox® by Elementis;
those sold under the name Nanogard WCD 2025® by Nanophase Technologies.

The coated zinc oxide pigments are, for example
those sold under the name Nanogard Zinc Oxide FN® by Nanophase Technologies (as a 40% dispersion in Finsolv TN®, C₁₂-C₁₅ alkyl benzoate);
those sold under the name Nanox Gel TN® by Elementis (ZnO dispersed at 55% in C₁₂-C₁₅ alkyl benzoate with hydroxystearic acid polycondensate).

The uncoated cerium oxide pigments may be, for example, those sold under the name Colloidal Cerium Oxide® by Rhône-Poulenc.

The uncoated iron oxide pigments are, for example, sold by Arnaud under the names Nanogard WCD 2002® (FE 45B®), Nanogard Iron FE 45 BL AQ, Nanogard FE 45R AQ® and Nanogard WCD 2006® (FE 45R®) or by Mitsubishi under the name TY-220®.

The coated iron oxide pigments are, for example, sold by Arnaud under the names Nanogard WCD 2008 (FE 45B FN)®, Nanogard WCD 2009® (FE 45B 556®), Nanogard FE 45 BL 345® and Nanogard FE 45 BL® or by BASF under the name Transparent Iron Oxide®.

Mention may also be made of mixtures of metal oxides, in particular of titanium dioxide and of cerium dioxide, including the equal-weight mixture of titanium dioxide and cerium dioxide coated with silica, sold by Ikeda under the name Sunveil A, or coated with alumina, silica and glycerol, such as the product M 211® sold by Sachtleben Pigments.

According to the invention, coated or uncoated titanium oxide pigments are particularly preferred, in particular titanium oxide pigments coated with alumina and aluminium stearate, such as the products Microtitanium Dioxide MT 100 TO, Microtitanium Dioxide MT-100 T V®, MT 100 TX®, MT 100 Z® and MT-01® from Tayca, the products Solaveil CT-10 W® and Solaveil CT 100® from Uniqema and the product Eusolex T-AVO® from Merck.

The inorganic UV-screening agents can be present in the composition according to the invention at a content ranging from 0.1% to 60% by weight and in particular from 5% to 30% by weight, relative to the total weight of the composition.

### Fillers

The compositions in accordance with the invention can also comprise at least one filler which makes it possible in particular to confer on them additional properties of improved stability, wear property, coverage and/or mattness.

The term “filler” should be understood as meaning colourless or white solid particles of any shape which are provided in an insoluble form and dispersed in the medium of the composition. They make it possible to confer body or firmness on the composition and/or softness and uniformity on the makeup.

The fillers can be inorganic or organic.

Preferably, they may be chosen from natural fillers or fillers of natural origin.

The term “natural compound” is understood to mean a compound which is obtained directly from the earth or the soil, or from plants or animals, via, if appropriate, one or more physical processes, such as, for example, milling, refining, distillation, purification or filtration.

The term “compound of natural origin” is understood to mean a natural compound which has undergone one or more additional chemical or industrial treatments, bringing about modifications which do not affect the essential qualities of this compound, and/or a compound predominantly comprising natural constituents which have or have not undergone transformations. Mention may be made, as nonlimiting example of additional chemical or industrial treatment bringing about modifications which do not affect the essential qualities of a natural compound, of those permitted by the controlling bodies, such as Ecocert (Reference system for biological and ecological cosmetic products, January 2003), or defined in recognized handbooks in the field, such as “Cosmetics and Toiletries Magazine”, 2005, Vol. 120, 9: 10.

The fillers used in the compositions according to the present invention can be of lamellar, globular, spherical or fibrous forms or of any other form intermediate between these defined forms.

The fillers according to the invention may or may not be surface-coated, and in particular they may be surface-treated with amino acids or any other substance which promotes the dispersion and the compatibility of the filler in the composition.

### a) Mineral Fillers

Mention may be made, as examples of inorganic fillers, of talcs, natural or synthetic micas, such as synthetic fluorphlogopites, silica, hydrophobic silica aerogels, hollow silica microspheres, kaolin, calcium carbonate, magnesium carbonate, hydroxyapatite, boron nitride, bismuth oxychloride, glass or ceramic microcapsules, or composites of silica and of titanium dioxide, such as the TSG® series sold by Nippon Sheet Glass.

Use will more preferentially be made of an inorganic filler chosen from natural or synthetic micas, hydrophobic silica aerogels, and mixtures thereof.

### Hydrophobic Silica Aerogel

According to one particularly preferred form, the composition of the invention also contains at least hydrophobic silica aerogel particles.

Silica aerogels are porous materials obtained by replacing (by drying) the liquid component of a silica gel with air.

They are generally synthesized by means of a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J. and Scherer G. W., Sol-Gel Science, New York, Academic Press, 1990.

The hydrophobic silica aerogel particles used in the present invention preferably have a specific surface area per unit mass (S/M) ranging from 500 to 1500 m²/g, preferably from 600 to 1200 m²/g and better still from 600 to 800 m²/g, and a size expressed as the volume-mean diameter (D[0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

The specific surface area per unit of weight can be determined by the nitrogen absorption method, known as the BET (Brunauer-Emmett-Teller) method, described in The Journal of the American Chemical Society, Vol. 60, page 309, February 1938 and corresponding to the international standard ISO 5794/1 (appendix D). The BET specific surface area corresponds to the total specific surface area of the particles under consideration.

The sizes of the silica aerogel particles can be measured by static light scattering using a commercial particle size analyzer of Mastersizer 2000® type from Malvern. The data are processed on the basis of the Mie scattering theory. This theory, which is exact for isotropic particles, makes it possible to determine, in the case of non-spherical particles, an “effective” particle diameter. This theory is described in particular in the publication by Van de Hulst, H. C., Light Scattering by Small Particles, Chapters 9 and 10, Wiley, New York, 1957.

According to one advantageous embodiment, the hydrophobic silica aerogel particles used in the present invention have a specific surface area per unit mass (S/M) ranging from 600 to 800 m²/g.

The silica aerogel particles used in the present invention can advantageously have a tapped density p ranging from 0.02 to 0.10 g/cm³, preferably from 0.03 to 0.08 g/cm³, in particular ranging from 0.05 to 0.08 g/cm³.

In the context of the present invention, this density can be assessed according to the following protocol, known as the tapped density protocol:

40 g of powder are poured into a measuring cylinder; the measuring cylinder is then placed on a Stav 2003® machine from Stampf Volumeter; the measuring cylinder is then subjected to a series of 2500 tapping actions (this operation is repeated until the difference in volume between two consecutive tests is less than 2%); the final volume Vf of tapped powder is then measured directly on the measuring cylinder. The tapped density is determined by the ratio w/Vf, in the case in point 40/Vf (Vf being expressed in cm³ and w in g).

According to one preferred embodiment, the hydrophobic silica aerogel particles used in the present invention have a specific surface area per unit volume SV ranging from 5 to 60 m²/cm³, preferably from 10 to 50 m²/cm³ and better still from 15 to 40 m²/cm³.

The specific surface per unit of volume is given by the relationship: SV=SM×ρ where is the tapped density expressed in g/cm3 and SM is the specific surface area per unit mass expressed in m²/g, as defined above.

Preferably, the hydrophobic silica aerogel particles used according to the invention have an oil absorption capacity, measured at the wet point, ranging from 5 to 18 ml/g, preferably from 6 to 15 m/g and better still from 8 to 12 ml/g.

The absorbing capacity measured at the wet point, denoted Wp, corresponds to the amount of oil that needs to be added to 100 g of particles in order to obtain a homogeneous paste.

It is measured according to the “wet point” method or the method for determining the oil uptake of a powder described in the standard NF T 30-022. It corresponds to the amount of oil adsorbed onto the available surface of the powder and/or absorbed by the powder by measuring the wet point, described below:

An amount m=2 g of powder is placed on a glass plate and then the oil (isononyl isononanoate) is added dropwise. After addition of 4 to 5 drops of oil to the powder, mixing is performed using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread over the glass plate without cracks or the formation of lumps. The volume Vs (expressed in ml) of oil used is then noted. The oil uptake corresponds to the ratio Vs/m.

The aerogels used according to the present invention are hydrophobic silica aerogels, preferably aerogels of silyl silica (INCI name: Silica Silylate).

The term “hydrophobic silica” means any silica of which the surface is treated with silylating agents, for example with halogenated silanes such as alkylchlorosilanes or silazanes, so as to functionalize the OH groups with silyl groups Si—Rn, for example trimethylsilyl groups.

As regards the preparation of hydrophobic silica aerogel particles surface-modified by silylation, reference may be made to U.S. Pat. No. 7,470,725.

Use will preferably be made of hydrophobic silica aerogel particles surface-modified with trimethylsilyl groups, preferably having the INCI name Silica Silylate.

As hydrophobic silica aerogels that may be used in the invention, an example that may be mentioned is the aerogel sold under the name VM-2260 or VM-2270 (INCI name: Silica Silylate) by Dow Corning, the particles of which have a mean size of about 1000 microns and a specific surface area per unit mass ranging from 600 to 800 m²/g.

Mention may also be made of the aerogels sold by Cabot under the references Aerogel TLD 201®, Aerogel OGD 2010 and Aerogel TLD 203®, Enova Aerogel MT 1100® and Enova Aerogel MT 1200®.

Use will preferably be made of the aerogel sold under the name VM-2270 (INCI name: Silica Silylate) by Dow Corning, the particles of which have an average size ranging from 5-15 microns and a specific surface area per unit mass ranging from 600 to 800 m²/g.

The silica aerogel particles, in particular of silylated silica aerogel, may be present in a composition according to the invention in a content ranging from 0.5% to 13%, preferably in a content ranging from 1% to 5% by weight and in particular ranging from 1% to 3% by weight relative to the total weight of the composition.

### b) Organic Fillers

Mention may be made, as examples of organic fillers, of micronized natural waxes; metal soaps derived from organic carboxylic acids having from 8 to 22 carbon atoms, preferably from 12 to 18 carbon atoms, for example zinc, magnesium or lithium stearate, zinc laurate or magnesium myristate; lauroyl lysine; or cellulose powders, such as that sold by Daito in the Cellulobeads® range.

Preferably, the filler(s) are present in the composition in a content ranging from 0.5% to 20% by weight, preferably from 1% to 15% by weight, more particularly from 3% to 10% by weight, relative to the total weight of the composition.

### Galling Agents

Depending on the viscosity of the composition which it is desired to obtain, one or more gelling agents which are hydrophilic, that is to say soluble or dispersible in water, and/or one or more gelling agents which are lipophilic, that is to say soluble or dispersible in water, can be incorporated in a composition of the invention.

Preferably, the hydrophilic gelling agents and/or the lipophilic gelling agents will be chosen from natural gelling agents or gelling agents of natural origin.

Mention may in particular be made, as hydrophilic gelling agents, of polysaccharide biopolymers, such as xanthan gum, guar gum, locust bean gum, acacia gum, scleroglucans, chitin and chitosan derivatives, carrageenans, gellans, alginates, celluloses, such as microcrystalline cellulose, cellulose gums, carboxymethylcellulose, hydroxymethykcellulose and hydroxypropylcellulose, and mixtures thereof.

As lipophilic gelling agents, mention may for example be made of lipophilic clays.

### Lipophilic-Clay

According to one particularly preferred form, the composition of the invention also contains at least one lipophilic clay.

The term “lipophilic clay” is intended to mean any clay that is liposoluble or lipodispersible in the oily phase of the composition.

Clay denotes a material based on hydrated silicates and/or aluminosilicates, of lamellar structure.

The clays can be natural or synthetic and they are rendered lipophilic by treatment with an alkylammonium salt, such as a C₁₀ to C₂₂ ammonium chloride, in particular stearalkonium chloride or distearyldimethylammonium chloride.

They may be chosen from bentonites, in particular bentonites, hectorites and montmorillonites, beidellites, saponites, nontronites, sepiolites, blotites, attapulgites, vermiculites and zeolites.

They are preferably chosen from hectorites and bentonites.

According to one particularly preferred form, use will be made of a lipophilic clay chosen from hydrophobically modified bentonites and hydrophobically modified hectorites, in particular modified with a C₁₀ to C₂₂ quaternary ammonium chloride, such as:
a bentonite modified with stearalkonium chloride, such as the commercial products sold under the name Claytone AF®, Garamite VT®, Tixogel® LG-M, Tixogel® MP 250 Tixogel® VZ and Tixogel® VZ-V XR, by BYK Additives Inc; or the commercial products sold under the name Viscogel® B3, Viscogel® B4, Viscogel® B7, Viscogel® B8, Viscogel® ED, Viscogel® GM, Viscogel® S4 and Viscogel® SD by Bentec S.P.A;
a bentonite modified with stearalkonlum chloride in the presence of at least propylene carbonate and of at least one oil, such as the commercial products Dub Velvet Gum® from Stearineries Dubois Fils,
Myglyol GEL T® from Cramer Oleo, Tixogel® CGT 6030, Tixogel® DBA 6060, Tixogel® FTN, Tixogel® FTN 1564, Tixogel® PM, Tixogel® LAN, Tixogel® LAN 1563 by BYK Additives Inc;
a hectorite modified with distearyldimethylammonium chloride (INCI name: Disteardimonium Hectorite) such as, for example, that sold under the name Bentone® 38V by Elementis Specialities;
a hectorite modified with distearyidimethylammonium chloride in the presence of at least propylene carbonate or triethyl citrate and of at least one oll, such as the products sold under the name Bentone® Gel DOA V, Bentone® Gel EUG V, Bentone® Gel IHD V, Bentone® Gel ISD V, Bentone® Gel MIO V® Bentone® Gel PTM VS Bentone® SS-71 V, Bentone® VS-5 PC V, Bentone® VS-5 by Elementis Specialities; the commercial products sold under the name Creagel Bentone CPS/Hectone CPS, Creagel Bentone ID/Hectone ID from Créations Couleurs; the commercial products sold under the name NS Gel DM1®, NS Gel PTIS®, NS MGel 1152® from Next Step Laboratories Stop.

The lipophilic clay(s) are present in the composition in concentrations ranging preferably from 0.1% to 5% by weight and more preferentially from 0.1% to 1% by weight relative to the total weight of the composition.

### Applications

According to one embodiment, a composition of the invention may advantageously be in the form of a composition for caring for the skin and/or keratin fibres, the body or the face, in particular the face.

According to another embodiment, a composition of the invention can advantageously be provided in the form of a composition for making up keratin materials, in particular the skin of the body or of the face, in particular of the face.

Thus, according to a sub-mode of this embodiment, a composition of the invention can advantageously be provided in the form of a base composition for makeup.

A composition of the invention can advantageously be provided in the form of a foundation.

Such compositions are in particular prepared according to the general knowledge of those skilled in the art.

The expressions “between . . . and . . . ”, and “ranging from . . . to . . . ” should be understood as meaning limits included, unless otherwise specified.

The invention is illustrated in greater detail by the examples presented below. Unless otherwise indicated, the amounts indicated are expressed as mass percentages.

### EXAMPLES 1 TO 6

Example 1 according to the invention and comparative examples 2 to 6 were prepared.

TABLE 1

| | | | Ex 2 | Ex 3 | Ex 4 | Ex 5 | Ex 6 |
|---|---|---|---|---|---|---|---|
| Phase | Ingredients | Ex. 1 | comp | comp | comp | comp | comp |
| A1 | PEG-30 Dipolyhydroxystearate | 5.00 | 5.00 | 5.00 | | | |
| | (Cithrol DPHS-SO-(MV)- Croda) | | | | | | |
| | Polyglyceryl-6 Polyricinoleate | | | | | 5.00 | |
| | (SY-Glyster CRS-75 ® - Sakamoto Yakuhin) | | | | | | |
| | Polyglyceryl-4 diisostearate | | | | | | 5.00 |
| | polyhydroxystearate sebacate | | | | | | |
| | (Isolan 7 ®- Evonik Goldschmidt) | | | | | | |
| | Dicaprylyl Carbonate | 11.80 | | 11.80 | 14.80 | 11.80 | 11.80 |
| | (Cetiol CC ® - BASF) | | | | | | |
| | Coco caprylate/caprate | 8.00 | 15.00 | | 8.00 | 8.00 | 8.00 |
| | (Cetiol C 5C ® - BASF) | | | | | | |
| | C15-C19 Alkanes | 3.00 | 7.00 | 7.00 | | 3.00 | 3.00 |
| A2 | Isododecane | 7.00 | 7.00 | 11.00 | 7.00 | 7.00 | 7.00 |
| | Silica Silylate | 0.10 | 0.10 | 0.10 | 0.10 | 0.10 | 0.10 |
| | (Dow Coming VM-2270 ® Aerogel Fine | | | | | | |
| | Particles) | | | | | | |
| | Disteardimonium Hectorite | 0.35 | 0.35 | 0.35 | 0.35 | 0.35 | 0.35 |
| | Bentone 38 VCG Rheological Additive ® - | | | | | | |
| | Elementis) | | | | | | |
| | Bismuth Oxychloride | 2.90 | 2.90 | 2.90 | 2.90 | 2.90 | 2.90 |
| | (Ronaflair LF 2000 ®-MERCK) | | | | | | |
| | Silica | 1.00 | 1.00 | 1.00 | 1.00 | 1.00 | 1.00 |
| B | Water | qs 100 | qs 100 | qs 100 | qs 100 | qs 100 | qs 100 |
| | Butylene Glycol | 3.00 | 3.00 | 3.00 | 3.00 | 3.00 | 3.00 |
| | Glycerol | 2.00 | 2.00 | 2.00 | 2.00 | 2.00 | 2.00 |
| | Hydrogenated Starch Hydrolysate | 0.50 | 0.50 | 0.50 | 0.50 | 0.50 | 0.50 |
| | (Neosorb 70/70B ® -Roquette) | | | | | | |
| | Pentylene Glycol | 1.00 | 1.00 | 1.00 | 1.00 | 1.00 | 1.00 |
| | Caprylyl glycol | 0.50 | 0.50 | 0.50 | 0.50 | 0.50 | 0.50 |
| | Cellulose gum | 0.38 | 0.38 | 0.38 | 0.38 | 0.38 | 0.38 |
| | (Blanose CMC 7M8SF ® -Ashland) | | | | | | |
| | Magnesium Sulfate | 0.66 | 0.66 | 0.66 | 0.66 | 0.66 | 0.66 |
| | Phenoxyethanol | 0.70 | 0.70 | 0.70 | 0.70 | 0.70 | 0.70 |
| C | Synthetic Fluorophlogopite | 1.43 | 1.43 | 1.43 | 1.43 | 1.43 | 1.43 |
| | Titanium dioxide (and) disodium stearoyl | 10.98 | 10.98 | 10.98 | 10.98 | 10.98 | 10.98 |
| | glutamate (and) aluminium hydroxide | | | | | | |
| | (NAI-TAO ®-77891 - Miyoshi Kasei) | | | | | | |
| | Iron oxides (and) Disodium Stearoyl | 0.84 | 0.84 | 0.84 | 0.84 | 0.84 | 0.84 |
| | Glutamate (and) Aluminium Hydroxide | | | | | | |
| | (NAI-R-800HP-10 ® - Miyoshi Kasei) | | | | | | |
| | Iron oxides (and) disodium stearoyl glutamate | 0.18 | 0.18 | 0.18 | 0.18 | 0.18 | 0.18 |
| | (and) aluminium hydroxide | | | | | | |
| | (NAI-YHP-10 ® - Miyoshi Kasei) | | | | | | |
| | Iron oxides (and) disodium stearoyl glutamate | 1.67 | 1.67 | 1.67 | 1.67 | 1.67 | 1.67 |
| | (and) aluminium hydroxide | | | | | | |
| | (NAI-BHP-10 ® - Miyoshi Kasei) | | | | | | |

### Preparation of the Compositions

The components of phase A1 were weighed out and the mixture stirred at 55° C. in order to obtain a homogeneous phase, then cooled to 30° C. The components of A2 were then weighed out and introduced into A1.

Aqueous phase B was emulsified in phase A with stirring for 20 minutes at 2500 rpm.

To prepare C, the pigments were milled in an air jet mill with 11.1%, relative to their weight, of synthetic fluorophlogopite. C was then introduced into the emulsion A+B with stirring for 10 minutes at 1500 rpm.

A panel of 14 individuals tested these foundations, on the face, with an amount of 0.075 ml of each example.

The following cosmetic criteria were evaluated on a scale of 0 to 15. The higher the mark, the greater the cosmetic effect.

TABLE 2

| | Example 1 | | | |
|---|---|---|---|---|
| | according | | | |
| | to the | Example 2 | Example 3 | Example 4 |
| Criteria evaluated | invention | comparative | comparative | comparative |
| Ease of spreading | 8 | 7.6 | 6.8 | 8.1 |
| during application | | | | |
| Thickness under | 7 | 8.1 | 6.7 | 7.9 |
| the fingers during | | | | |
| application | | | | |
| Drying speed | 6.3 | 5 | 5.2 | 6.1 |
| Dry skin | 5 | 4.8 | 6.1 | 6.2 |
| 5 minutes after | | | | |
| application | | | | |
| Oily skin | 10.5 | 12 | 10 | 10.2 |
| 5 minutes after | | | | |
| application | | | | |
| Slippery skin | 5 | 4.1 | 5.3 | 5.2 |
| 5 minutes after | | | | |
| application | | | | |
| Tacky skin | 10 | 10.3 | 11.3 | 9.9 |
| 5 minutes after | | | | |
| application | | | | |
| Soft skin | 7.9 | 7.8 | 6.7 | 6.8 |
| 5 minutes after | | | | |
| application | | | | |

The results show that Example 1 corresponded to the preferred composition of the testers. It resulted in a fine deposit which was easy to spread, with a soft feel, emollient and non-tacky.

Composition 2 was judged to be the least preferred; it was judged to be thick, taking too long to put on and making it difficult to put on makeup. It was judged to have an oily and greasy deposit. Composition 3 was judged to drag and to be difficult to spread, with a deposit that was dry, not soft and was tacky. Composition 4 was judged to be non-fine, with a deposit that was dry and not soft.

Contrary to Example 1 of the invention, Compositions 5 and 6 outside the invention exhibited phase separation at the surface, which appeared as early as after 1 month at ambient temperature, and was accentuated when the emulsion was stored at a higher temperature, for example at 37° C.

1. Composition in the form of a silicone-free, water-in-oil emulsion comprising, in a physiologically acceptable medium:
A) a continuous oily phase comprising
i) at least one C12-C22 dialkyl carbonate;
ii) at least one mixture of esters of C8-C10 carboxylic acid(s) and of fatty alcohols derived from coconut oil;
iii) a mixture of C15-C19 alkanes; and
iv) at least one volatile hydrocarbon-based oil;
B) an aqueous phase dispersed in said oily phase; and
C) at least one emulsifying non-ionic surfactant of the polyoxyethylenated glycol fatty acid ester polymer type.

2. Composition according to claim 1, in which the at least one C12-C22 dialkyl carbonate is selected from the group consisting of:
di-2-ethylhexyl carbonate;
dicaprylyl carbonate;
a mixture of C14-C15 dialkyl carbonates (INCI name: C14-C15 Dialkyl Carbonate);
dipropylheptyl carbonate; and
mixtures thereof.

3. Composition according to claim 1, in which the at least one C12-C22 dialkyl carbonate is present in contents ranging from 5% to 20% by weight relative to the total weight of said composition.

4. Composition according to claim 1, in which the at least one mixture of esters of C8-C10 carboxylic acid(s) and of fatty alcohols derived from coconut oil is a mixture of caprylic acid (C8) and capric acid (C10) and of coconut oil fatty alcohols, having the INCI name Coco Caprylate/Caprate.

5. Composition according to claim 1, in which the at least one mixture of esters of C8-C10 carboxylic acid(s) and of fatty alcohols derived from coconut oil is present in contents ranging from 2% to 20% by weight relative to the total weight of said composition.

6. Composition according to claim 1, in which the at least one volatile hydrocarbon-based oil is selected from the group consisting of volatile linear alkanes comprising 6 to 14 carbon atoms.

7. Composition according to claim 6, in which the at least one volatile hydrocarbon-based oil is selected from the group consisting of:
a mixture of n-dodecane and n-tetradecane;
a mixture of linear C9-C12 alkanes; and
a mixture of undecane and tridecane.

8. Composition according to claim 1, in which the at least one volatile hydrocarbon-based oil is selected from the group consisting of branched C8-C16 alkanes.

9. Composition according to claim 1, in which the at least one volatile hydrocarbon-based oil is present in contents ranging from 2% to 20% by weight relative to the total weight of said composition.

10. Composition according to claim 1, in which the amount of the mixture of linear C15-C19 alkanes is less than or equal to 7% by weight relative to the total weight of the composition.

11. Composition according to claim 1, in which the mixture of linear C15-C19 alkanes is present in contents ranging from 1% to 12% by weight relative to the total weight of the composition.

12. Composition according to claim 1, in which the at least one emulsifying non-ionic surfactant of the polyoxyethylenated glycol fatty acid ester polymer type is polyethylene glycol dipolyhydroxystearate containing 30 mol of ethylene oxide, having the INCI name: PEG-30 Polyhydroxystearate.

13. Composition according to claim 1, in which the at least one emulsifying non-ionic surfactant of the polyoxyethylenated glycol fatty acid ester polymer type is present in the composition in contents ranging from 0.1% to 10% by weight relative to the total weight of the composition.

14. Composition according to claim 1, further comprising water in a content ranging from 20% to 50% by weight relative to the total weight of the composition.

15. Composition according to claim 1, further additionally comprising at least one pulverulent colorant.

16. Composition according to claim 15, in which the at least one pulverulent colorant is selected from the group consisting of mineral pigments, organic pigments, pearlescent agents, and mixtures thereof.

17. Composition according to claim 15, wherein the at least one pulverulent colorant is selected from the group consisting of metal oxides, which are or coated or uncoated, and mixtures thereof.

18. Composition according to claim 15, in which the at least one pulverulent colorant is present in a content ranging from 0.5% to 30% by weight relative to the total weight of the composition.

19. Composition according to claim 1, further claims, also comprising at least one additive selected from the group consisting of:
active agents;
ceramides;
inorganic UV-screening agents;
colorants;
fillers;
hydrophilic gelling agents;
lipophilic gelling agents;
fragrances;
preserving agents;
and mixtures thereof.

20. Composition according to claim 1, further, comprising at least one inorganic UV-screening agent selected from the group consisting of coated or uncoated metal oxide pigments.

21. Composition according to claim 1, further comprising at least one filler selected from the group consisting of hydrophobic silica aerogel particles.

22. Composition according to claim 1, further comprising at least one lipophilic clay.

23. Process for coating keratin materials comprising applying to the keratin materials a composition according to claim 1.
